SEQUENCE LISTING (3 sequences)

SEQ ID NO: 1 - protein, 17 aa, Artificial Sequence
  Synthetic polypeptide
KGVSLSYRXRYSLSVGK

SEQ ID NO: 2 - protein, 19 aa, Artificial Sequence
  Synthetic polypeptide
KGVSLSYRCXCRYSLSVGK

SEQ ID NO: 3 - protein, 10 aa, Artificial Sequence
  Synthetic polypeptide
KGVSLSYRCX

(19) United States
(12) Patent Application Publication
(10) Pub. No.: US 2017/0143787 A1
(43) Pub. Date: May 25, 2017

(54) CHEMOKINE RECEPTOR ANTAGONIST AND ITS COMBINATIONAL THERAPY

(71) Applicants: Pertinax Therapeutics Inc., Markham (CA); Microconstants China Inc., Beijing (CN)

(72) Inventors: Beverly James Incledon, George Town, Grand Cayman (KY); Yiu-Chung Lee, Markham (CA); Qicheng David Yang, San Diego, CA (US)

(21) Appl. No.: 15/119,931

(22) PCT Filed: Feb. 19, 2014
(86) PCT No.: PCT/CN2014/072239

§ 371 Date: Aug. 18, 2016

(51) Int. Cl. A61K 38/10; A61K 45/06; A61N 5/10; A61K 38/08

(52) U.S. Cl. CPC A61K 38/10; A61K 38/08; A61K 45/06; A61N 5/10; A61N 5/1001

(57) ABSTRACT

A method for treating various types of cancer/tumor by administering the combination of SDF-1 antagonist, in particular, that specifically binds to human CXCR4, and another chemotherapeutic agent, or a radiation therapy, during an embolization therapy. Such combination therapies exhibit synergistic effects compared to the treatment with either agent alone. Thus, the method is particularly beneficial for cancer patients who have low tolerance to the side effects caused by high dosages required for the treatment by either agent alone.

### FIELD OF THE INVENTION

The present invention belongs to the pharmaceutical field. In particular, the present invention relates to use of a peptide as a chemokine receptor antagonist for treating cancer or reducing or halting tumor growth in a subject during an embolization therapy.

### BACKGROUND OF THE INVENTION

Cytokines are soluble proteins secreted by a variety of cells including monocytes or lymphocytes that regulate immune responses. Chemokines are a superfamily of chemoattractant proteins. Chemokines regulate a variety of biological responses and they promote the recruitment of multiple lineages of leukocytes and lymphocytes to a body organ tissue. Chemokines may be classified into two families according to the relative position of the first two cysteine residues in the protein. In one family, the first two cysteines are separated by one amino acid residue, e.g., the CXC chemokines, and in the other family the first two cysteines are adjacent, e.g., the CC chemokines.

The molecular targets for chemokines are cell surface receptors. One such receptor is CXC chemokine receptor 4 (CXCR4), which is a seven-transmembrane protein, coupled to G1 and was previously called LESTR (Loetscher, M., Geiser, T., O'Reilly, T., Zwahlen, R., Baggionlini, M., and Moser, B., (1994) J. Biol. Chem, 269, 232-237), HUMSTR (Federsppiel, B., Duncan, A. M. V., Delaney, A., Schappert, K., Clark-Lewis, I., and Jirik, F. R. (1993) Genomics 16, 707-712) and Fusin (Feng, Y., Broeder, C. C., Kennedy, P. E., and Berger, E. A. (1996) HIV-1 entry cofactor: Functional cDNA cloning of a seven-transmembrane G protein-coupled receptor, Science 272, 872-877). CXCR4 is widely expressed on cells of hemopoietic origin, and is a major co-receptor with CD4.sup.+ for human immunodeficiency virus 1 (HIV-1) (Feng, Y., Broeder, C. C., Kennedy, P. E., and Berger, E. A. (1996) HIV-1 entry cofactor: Functional cDNA cloning of a seven-transmembrane G protein-coupled receptor, Science 272, 872-877).

Currently, the only known natural ligand for CXCR4 is stromal cell derived factor one (SDF-1). Stromal cell derived factor-1α (SDF-1α) and stromal cell derived factor-1β (SDF-1β) are closely related members (together referred to herein as SDF-1). The native amino acid sequences of SDF-1α and SDF-1β are known, as are the genomic sequences encoding these proteins (U.S. Pat. No. 5,563,048 issued 8 Oct. 1996, and U.S. Pat. No. 5,756,084 issued 26 May 1998).

SDF-1 is functionally distinct from other chemokines in that it is reported to have a fundamental role in the trafficking, export and homing of bone marrow progenitor cells (Aiuti, A., Webb, I. J., Bleul, C., Springer, T., and Guierrez-Ramos, J. C., (1996) J. Exp. Med. 185, 111-120 and Nagasawa, T., Hirota, S., Tachibana, K., Takakura N., Nishikawa, S.-I., Kitamura, Y., Yoshida, N., Kikutani, H., and Kishimoto, T., (1996) Nature 382, 635-638). SDF-1 is also structurally distinct in that it has only about 22% amino acid sequence identity with other CXC chemokines (Bleul, C. C., Fuhlbrigge, R. C., Casasnovas, J. M., Aiuti, A., and Springer, T. A., (1996) J. Exp. Med. 184, 1101-1109). SDF-1 appears to be produced constitutively by several cell types, and particularly high levels are found in bone-marrow stromal cells (Shirozu, M., Nakano, T., Inazawa, J., Tashiro, K., Tada, H. Shinohara, T., and Honjo, T., (1995) Genomics, 28, 495-500 and Bleul, C. C., Fuhlbrigge, R. C., Casasnovas, J. M., Aiuti, A., and Springer, T. A., (1996) J. Exp. Med. 184, 1101-1109). A basic physiological role for SDF-1 is implied by the high conservation of the SDF-1 sequences between species. In vitro, SDF-1 stimulates chemotaxis of a wide range of cells including monocytes and bone marrow derived progenitor cells (Aiuti, A., Webb, I. J., Bleul, C., Springer, T., and Guierrez-Ramos, J. C., (1996) J. Exp. Med. 185, 111-120 and Bleul, C. C., Fuhlbrigge, R. C., Casasnovas, J. M., Aiuti, A., and Springer, T. A., (1996) J. Exp. Med. 184, 1101-1109). Particularly notable is its ability to stimulate a high percentage of resting and activated T-lymphocytes (Bleul, C. C., Fuhlbrigge, R. C., Casasnovas, J. M., Aiuti, A., and Springer, T. A., (1996) J. Exp. Med. 184, 1101-1109 and Campbell, J. J., Hendrick, J., Zlotnik, A., Siani, M. A., Thompson, D. A., and Butcher, E. C., (1998) Science, 279 381-383).

The 3-dimensional crystallographic structure of SDF-1 has been described (Crump, M., Gong J.-H., Loetscher, P., Rajarathnam, K., Amara, A., Arenzana-Seisdedos, F., Virelizier, J.-L., Baggiolini, M., Sykes, B. D., and Clark-Lewis, I., (1997) EMBO J., 16, 6996-7007). Structure-activity analysis of SDF-1 (Crump, M., Gong J.-H., Loetscher, P., Rajarathnam, K., Amara, A., Arenzana-Seisdedos, F., Virelizier, J.-L., Baggiolini, M., Sykes, B. D., and Clark-Lewis, I., (1997) EMBO J., 16, 6996-7007) indicates that although N-terminal residues 1-8 or 1-9 are involved in receptor binding, the 1-8 and 1-9 peptides alone exhibited no in vitro activity indicative of receptor binding, supporting a reported conclusion that the peptides do not assume the conformation necessary for binding to the receptor. This result was taken to imply that the remainder of the protein scaffold, and/or various consensus receptor binding sites elsewhere in the protein are important for mediating the conformational requirements for N-terminal binding to the receptor (Crump, M., Gong J.-H., Loetscher, P., Rajarathnam, K., Amara, A., Arenzana-Scisdcdos, F., Virelizier, J.-L., Baggiolini, M., Sykes, B. D., and Clark-Lewis, I., (1997) EMBO J., 16, 6996-7007). Based on these results, a two-site model has been proposed for SDF-1 binding to CXCR4, involving two binding sites in residues 1-17, an N-terminal site and an upstream RFFESH site (Crump, M., Gong J.-H., Loetscher, P., Rajarathnam, K., Amara, A., Arenzana-Seisdedos, F., Virelizier, J.-L., Baggiolini, M., Sykes, B. D., and Clark-Lewis, I., (1997) EMBO J., 16, 6996-7007). The two putative binding sites have been characterised by the sequence: KPVSLSYR-CPC-RFFESH, in which the two putative binding sites are joined by the CXC motif that characterises the whole CXC chemokine family (Crump, M., Gong J.-H., Loetscher, P., Rajarathnam, K., Amara, A., Arenzana-Seisdedos, F., Virelizier, J.-L., Baggiolini, M., Sykes, B. D., and Clark-Lewis, I., (1997) EMBO J., 16, 6996-7007). These two putative binding regions have been identified as being important in other CC and CXC chemokines (Crump, M., Gong J.-H., Loetscher, P., Rajarathnam, K., Amara, A., Arenzana-Seisdedos, F., Virelizier, J.-L., Baggiolini, M., Sykes, B. D., and Clark-Lewis, I., (1997) EMBO J., 16, 6996-7007 and Crump, M., Gong J.-H., Loetscher, P., Rajarathnam, K., Amara, A., Arenzana-Seisdedos, F., Virelizier, J.-L., Baggiolini, M., Sykes, B. D., and Clark-Lewis, I., (1997) EMBO J., 16, 6996-7007). This is consistent with the finding that although N-terminal regions of a wide variety of chemokines are critical for receptor activation, N-terminal peptides of chemokines other than SDF-1 have been reported to lack receptor binding activity and not to be receptor agonists (Crump, M., Gong J.-H., Loetscher, P., Rajarathnam, K., Amara, A., Arenzana-Seisdedos, F., Virelizier, J.-L., Baggiolini, M., Sykes, B. D., and Clark-Lewis, I., (1997) EMBO J., 16, 6996-7007 and Crump, M., Gong J.-H., Loetscher, P., Rajarathnam, K., Amara, A., Arenzana-Seisdedos, F., Virelizier, J.-L., Baggiolini, M., Sykes, B. D., and Clark-Lewis, I., (1997) EMBO J., 16, 6996-7007).

Consistent with the fact that CXCR4 is a major co-receptor for HIV-1, SDF-1 blocks HIV-1 entry into CD4.sup.+ cells Oberlin, E., Amara, A., Bachelerie, F., Bessia, C., Virelizier, J.-L., Arenzana-Seisdedos, F., Schwartz, O., Heard, J.-M., Clark-Lewis, I., Legler, D. F., Loetscher, M., Baggiolini, M., and Moser, B., (1996) Nature, 382, 833-835 and Bleul, C. C., Farzan, M., Choe, H., Parolin, C., Clark-Lewis, I., Sodroksi, J., and Springer, T. A., (1996) Nature, 382, 829-833). Efforts have been made to identify SDF-1 derived peptides that interfere selectively with HIV entry, but not with SDF-1 signalling (Heveker, N. et al., 1998, Current Biology 8(7):369-376). A wide range of potential CXCR4 binding fragments of SDF-1 have been proposed for use in blocking HIV infection (WO 9728258, published 7 Aug. 1997; WO 9804698, published 5 Feb. 1998). As these references make clear, the anti-HIV activity of SDF-1, or fragments of SDF-1, does not depend on antagonism of the CXCR4 receptor.

Interferon gamma is an important cytokine that is released by activated T-lymphocytes (T-cells) and acts as a potent immunomodulator. Interferon gamma production by T-cells in vivo may cause other cells in the body to release additional cytokines, enzymes and antibodies that are capable of modulating many aspects of an immune response. Agents which affect the ability of activated T-cells to produce interferon gamma are characterized as immunomodulators.

Autoimmune diseases are a group of illnesses generally understood to be caused by the over-production of cytokines, lymphotoxins and antibodies by white blood cells, including in particular T-cells. During an autoimmune reaction, T-cells are understood to release chemical mediators such as interferon gamma which lead to the development of pathological symptoms of autoimmune reaction. A treatment for autoimmune diseases may therefore involve the use of agents capable of inhibiting release of interferon gamma from T-cells. Such autoimmune diseases may include, for example, Multiple Sclerosis (MS), Guillain-Barre Syndrome, Amotrophic Lateral Sclerosis, Parkinson's disease, Alzheimer's disease, Gout, Lupus, and any other human illnesses that T-cells play a major role in.

Interferon beta is a cytokine that has found to have therapeutic application in the treatment of a variety of autoimmune diseases. In autoimmune diseases such as MS, the activation of Th1 type T-cells is thought to be a primary component of the autoimmune response. In MS, the autoimmune response attacks the myelin sheath neuronal axons. One of the classical markers of Th1 cell activation is the production of interferon gamma. In the development of interferon beta as a therapeutic agent for the treatment of MS, studies were conducted to demonstrate the ability of interferon beta to decrease the rate of production of interferon gamma from lymphocytes in vitro (Ann. Neurol. 1998; 44: 27-34 and Neurology 1998; 50: 1294-1300). The reduction of interferon gamma release by treatment with interferon beta is an indication of the effectiveness of interferon beta in the treatment of MS. There is a continuing need for other agents that inhibit the production of interferon gamma, particularly agents for use in the treatment of autoimmune disease, including agents that may work synergistically to enhance the effect of existing agents such as interferon beta.

Solid tumour growth is generally angiogenesis (neovascularization)-dependent, and angiogenesis inhibitors have therefore been used as agents for the treatment of solid tumours and metastasis. Endothelial cells (EC) in the vasculature play an essential role in angiogenesis, and there is accordingly a need for therapeutic agents that target this activity. The proliferation, migration and differentiation of vascular endothelial cells during angiogenesis is understood to be modulated in both normal and disease states by the complex interactions of a variety of chemokines and chemokine receptors. CXCR4 is expressed on vascular EC, and in such cells is reportedly the most abundant receptor amongst all examined chemokine receptors (Gupta, et al, 1998).

Prior art (U.S. patent application Ser. No. 11/136,097) indicated that CXCR4 receptor ligand, SDF-1 polypeptide antagonists with amino acid sequence similar to SEQ ID NO.1, SEQ ID NO.2, or SEQ ID NO.3 exhibits anti-angiogenesis and anti-tumor activities in various embodiments.

### SUMMARY OF THE INVENTION

Here, the inventors demonstrate that the combination of these SDF-1 antagonists with hepatic artery ligation procedure in rats, which is similar to embolization therapy in human, inhibits the growth of the liver cancer xenografts.

Accordingly, the present invention relates to use of a peptide as a chemokine receptor antagonist, optionally in combination with one or more other chemotherapeutic agent(s) and/or a radiation therapy, in preparation of a medicament for treating cancer or reducing or halting tumor growth in a subject during an embolization therapy,

wherein the peptide specifically binds to human CXCR4.

The present invention also relates to a method of treating cancer or reducing or halting tumor growth in a subject, comprising administering to the subject a peptide, optionally in combination with one or more other chemotherapeutic agent(s) and/or a radiation therapy during an embolization therapy, wherein the peptide specifically binds to human CXCR4.

Preferably, the peptide further comprises:
(1) an amino acid sequence represented by SEQ ID NO.1, 2 or 3, or
(2) an amino acid sequence obtained by one or several conservative substitutions in SEQ ID NO.1, 2 or 3.

Preferably, the peptide may consist of the amino acid sequence of SEQ ID NO.1, 2 or 3, or their variants resulted from one or several conservative substitutions (e.g., 1, 2, 3, 4, 5, 6, 7, or 8 conservative substitutions).

Preferably, the subject is a human.

In an embodiment, the chemotherapeutic agent is at least one selected from the group consisting of an anti-mitotic agent, platinum-based chemotherapeutic agent, receptor tyrosine kinase inhibitor, pyrimidine analogue, topoisomerase inhibitor, and adjuvant.

Preferably, the anti-mitotic agent is docetaxel or paclitaxel, or a pharmaceutically acceptable analogue or salt thereof; the platinum-based chemotherapeutic agent is cisplatin, carboplatin, iproplatin, or oxaliplatin, or a pharmaceutically acceptable salt thereof; the receptor tyrosine kinase inhibitor is sorafenib, sunitinib, or pazopanib, or a pharmaceutically acceptable salt thereof; the pyrimidine analogue is gemcitabine, 5-FU, or capecitabine, or a pharmaceutically acceptable salt thereof; the topoisomerase inhibitor is irinotecan, topotecan, camptothecin, or lamellarin D, or a pharmaceutically acceptable salt thereof; and the adjuvant is folinic acid, or a pharmaceutically acceptable salt thereof.

Alternatively, the chemotherapeutic agent is a combination of 5-FU, folinic acid and oxaliplatin; 5-FU, folinic acid and irinotecan; capecitabine and oxaliplatin; or cisplatin and gemcitabine.

In an embodiment, the radiation is X-rays, gamma rays or charged particles delivered either by a machine outside the body or from a radioactive material placed in the body near the cancer/tumor cells. Preferably, the radioactive material is radioactive iodine.

In an embodiment, the cancer is selected from the group consisting of ovarian cancer, uterus cancer, breast cancer, lung cancer, liver cancer, colorectal cancer, bladder cancer, renal cancer, prostate cancer, pancreatic cancer, stomach cancer, bone cancer, skin cancer, visceral cancers, adrenal and pheochromocytomas, leukemia, and malignant soft tissue sarcoma.

In an embodiment, the peptide and the chemotherapeutic agent are administered concurrently. In another embodiment, the peptide and the chemotherapeutic agent are administered sequentially.

In an embodiment, the embolization therapy is trans-arterial embolization (TAE), trans-arterial chemoembolization (TACE), or radioembolization (RE).

Specifically, the present invention provides a new therapeutic use for CXCR4 antagonists/inhibitors in an embolization therapy, i.e., the present invention provides a combinational therapy. In certain embodiments, CXCR4 antagonists may be used therapeutically as follows, or to manufacture a medicament for such therapeutic treatments: treatment of cancer, and regulation of angiogenesis. In some aspects of the invention, CXCR4 inhibitors may be used, with or without another chemotherapy agent during the EMBOLIZATION THERAPY procedure. The invention provides corresponding methods of medical treatment, in which a therapeutic dose of a CXCR4 antagonist is administered in a pharmaceutically acceptable formulation. Accordingly, the invention also provides pharmaceutical compositions comprising a CXCR4 antagonist and a pharmaceutically acceptable excipient or carrier, conventional in the field, or as described below. The pharmaceutical composition may advantageously be soluble in an aqueous solution at a physiologically acceptable pH.

The CXCR4 antagonists for use in the invention may be peptide compounds comprising a substantially purified peptide fragment, modified fragment, analog or pharmaceutically acceptable salt of SDF-1. In some embodiments, the peptide compound may comprise an N-terminal amino acid sequence: KGVSLSYRC-X (SEQ ID NO.3) wherein X is selected from the group consisting of hydrogen and a polypeptide homologous to at least a portion of SDF-1.

In a further embodiment, the peptide compound may comprise a dimerized N-terminal amino acid sequence (represented here with the second dimer written from the carboxyl to the amino terminus): KGVSLSYR-X-RYSLSVGK (SEQ ID NO.1, named CTCE-9908) wherein X may be a lysine amino acid wherein both the α- and ε-amino groups are associated with amide bond formation with arginine residue and the lysyl carboxl group may be protected through acetate acid reaction. In yet another embodiment, the peptide compound may further comprise a dimerized, N-terminal amino acid (represented here with the second dimer written from the carboxyl to the amino terminus): KGVSLSYRC-X-CRSLSVGK (SEQ ID NO.2), wherein X may be a lysine amino acid wherein both the α- and ε-amino groups are associated with amide bond formation with cysteine residue and the lysyl carboxgl group may be protected through acetate acid reaction. Alternatively, in the aforementioned dimerized peptide compounds, X may be any bridge-forming moiety that covalently links peptides so that a plurality of peptides are joined by the bridge to provide a plurality of N-terminals in the compounds.

In accordance with one aspect of the present invention, antagonists of CXCR4 may be used therapeutically to regulate angiogenesis and cell growth in human pathological diseases including cancers such as lymphoma and carcinoma, as well as restenosis. In one embodiment, as exemplified herein, the peptide CXCR4 antagonist has been used to inhibit angiogenesis and tumor growth in mouse models of mammalian cancers.

In various aspects, the present invention utilizes CXCR4 antagonists. In some embodiments, the CXCR4 antagonists for use in the invention may be substantially purified peptide fragments, modified peptide fragments, analogues or pharmaceutically acceptable salts of either SDF-1α or SDF-1β. SDF-1 derived peptide antagonists of CXCR4 may be identified by known physiological assays and a variety of synthetic techniques (such as disclosed in Crump et al., 1997, The EMBO Journal 16(23) 6996-7007; and Heveker et al., 1998, Current Biology 8(7): 369-376; each of which are incorporated herein by reference). Such analogs of SDF-1 include homologs of native SDF-1, such as naturally occurring isoforms or genetic variants, or polypeptides having substantial sequence similarity to SDF-1, such as 40% sequence identity, 60% sequence identity or preferably 80% sequence identity to at least a portion of the native SDF-1 sequence, provided they have CXCR4 antagonistic activity. In some embodiments, chemically similar amino acids may be substituted for amino acids in the native SDF-1 sequence (to provide conservative amino acid substitutions).

It is anticipated that SEQ ID NO.1, 2 or 3 may have variants by addition, deletion or substitution of one or several amino acid residue(s), without any adverse effects on their activities as chemokine receptor antagonists, especially as antagonists of CXCR4. There are various conservative substitutions of one, two, three, or even four, up to eight, amino acid residues in SEQ ID NO.1, 2 or 3. For example, the conservative substitution may occur among acidic amino acids (e.g., Glu and Asp), alkaline amino acids (e.g., Lys and Arg), hydroxyl amino acids (e.g., Ser and Thr), or aromatic amino acids (e.g., Phe, Trp and Tyr). In particular, X of SEQ ID NO.1 or 2 may be Lys or Arg.

In particular embodiments, a preferred range for therapeutically or prophylactically effective amounts of CXCR4 antagonist may be 0.01 nM-0.1M, particularly 0.1 nM-0.05M, more particularly 5 nM-15 mM and most particularly 0.1 mM-10 mM. It is to be noted that dosage values may vary with the severity of the condition to be alleviated, especially for multiple sclerosis. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the composition.

The amount of active compound in the composition may vary according to factors such as the disease state, age, gender, and body weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention is dictated by and directly dependent on (a) the unique characteristics of the active compound and the particular therapeutic effect to be achieved, and (b) the limitations inherent in the art of compounding such an active compound for the treatment of sensitivity in individuals.

As used herein, the term “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds (e.g., one or more of other chemotherapeutic agents) can also be incorporated into the compositions.

Peptides of the present invention may be chemically synthesized using standard techniques such as those described in Bodansky, M. Principles of Peptide Synthesis, Springer Verlag, Berlin (1993) and Grant, G. A. (ed.). Synthetic Peptides: A User's Guide, W.H. Freeman and Company, New York (1992) (all of which are incorporated herein by reference). Automated peptide synthesizers are commercially available (e.g., Advanced ChemTech Model 396; Milligen/Biosearch 9600).

In addition, peptides of the present invention may be prepared according to standard recombinant DNA techniques using a nucleic acid molecule encoding the peptide. A nucleotide sequence encoding the peptide can be determined using the genetic code and an oligonucleotide molecule having this nucleotide sequence can be synthesized by standard DNA synthesis methods (e.g., using an automated DNA synthesizer) Alternatively, a DNA molecule encoding a peptide compound can be derived from the natural precursor protein gene or cDNA (e.g., using the polymerase chain reaction (PCR) and/or restriction enzyme digestion) according to standard molecular biology techniques.

### BRIEF DESCRIPTION OF FIGURES

FIG. 1: SEQ ID NO.1 peptide dimer (also called CTCE-9908) in combination with hepatic artery ligation (HAL+ CTCE-9908) decreased tumor size in hepatocellular carcinoma. Rats that were treated with CTCE-9908 in combination with hepatic artery ligation (HAL+ CTCE-9908) showed a tumor regression of up to 80%. This is evident both by luminescence assessment and at necropsy.

FIG. 2: CTCE-9908 in combination with hepatic artery ligation reduced the number of circulating hemangiocytes. Compared to hepatic artery ligation alone, treatment with HAL and CTCE-9908 at 12.5 mg/kg reduced CXCR4+Flt-1+ hemangiocytes by about 80% to 90%. Reduction in recruitment of these cells may contribute to the shrinkage of the tumor.

FIG. 3: CTCE-9908 with or without hepatic artery ligation (HAL) significantly increased survival of the rat model of hepatocellular carcinoma (HCC). HAL alone and sham surgery did not significantly affect survival of rats with HCC. CTCE-9908 treatment alone at 25 mg/kg significantly increased the survival compared to HAL alone and sham surgery. CTCE-9908 at both 12.5 mg/kg and 25 mg/kg in combination with HAL further increased the survival compared to CTCE-9908 alone.

### EMBODIMENTS OF THE INVENTION

The present invention will be further illustrated below by reference to the following Examples. It is to be understood that this invention is not limited to the Examples.

### Example 1

This example shows the inhibitory effects of CXCR4 antagonists on tumor growth using rat hepatic artery ligation models.

The CXCR4 antagonists used was the short peptide dimer antagonist CTCE-9908 (SEQ ID NO.1). A rat orthotopic hepatocellular carcinoma (HCC) model was established by injection of a rat HCC cell line, CRL-1601, into the left lobe of the liver of Buffalo rats. Since the rat HCC cell line was generated from Buffalo rats, in vivo formed tumor in the Buffalo rats can mimic the clinical setting. Two weeks after tumor cell injection, rats were randomized into the following groups:
1) Sham operation, n=6;
2) Hepatic artery ligation, n=6;
3) CTCE-9908 at 12.5 mg/kg/day, n=6;
4) CTCE-9908 at 25 mg/kg/day, n=6;
5) Hepatic artery ligation combined with CTCE-9908 at 12.5 mg/kg/day, n=6;
6) Hepatic artery ligation combined with CTCE-9908 at 25 mg/kg/day, n=6.

Natural hypoxia commonly occurs in large HCC tumor nodules. The procedure of hepatic artery ligation is to mimic the therapeutic approach of EMBOLIZATION PROCEDURE, the most commonly used treatment for unresectable HCC. This therapeutic approach on one hand leads to tumor cell necrosis, on the other hand it induces a more severe hypoxic condition to the residual tumor cells, leading to the release of pro-angiogenic factors and subsequent tumor re-growth. Therefore, in this proposed study, we apply CTCE-9908 with or without hepatic artery ligation to explore the effects of this drug, either used alone or combined with ischemic hypoxia for the treatment of HCC. The CRL-1601 HCC cell line is CXCR4 negative. Therefore, CTCE-9908 would not have a direct effect on the tumor cells themselves.

The first group (control) received sham operation followed by water for irrigation. The second, fifth and sixth groups were given hepatic artery ligation. The third and fifth groups were given CTCE-9908 at a dose of 12.5 mg/kg subcutaneously every day. The fourth and sixth groups were given CTCE-9908 at a dose of 25 mg/kg subcutaneously every day. Treatment was continued for 4 weeks. One week after the last treatment, both control and treated groups were sacrificed. The dimension of the primary tumour was measured along with liver function. The microvessel density, proliferation index of tumor cells, apoptotic state and morphology of tumour cells in primary tumour and metastases were analysed by immunohistochemistry. The lungs were fixed with formalin, embedded in paraffin, sliced serially and mounted on slides. The number of metastatic tumour loci was analysed. Plasma SDF-1 was analyzed by ELISA. Blood was collected for analysis of hemangiocytes and endothelial progenitor cells by flow cytometry.

This study examined a rat model of hepatocellular carcinoma (HCC) that mimics transarterial chemoembolization in the clinical setting. Rat HCC were implanted into the left lobe of the liver and allowed to grow for 2 weeks at which time the tumor mass were greater than 10 mm in diameter. Rats were then given hepatic artery ligation (HAL), and CTCE-9908 treatment at 12.5 mg/kg/day or 25 mg/kg/day for four weeks. One week after the last CTCE-9908 treatment, the rats were sacrificed and assessments of the tumor as well as the circulating cells were made by luminescence and flow cytometry, respectively.

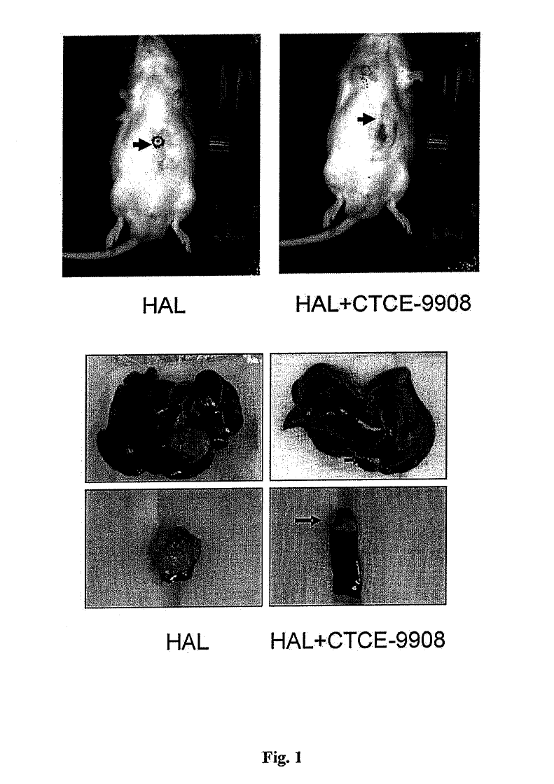

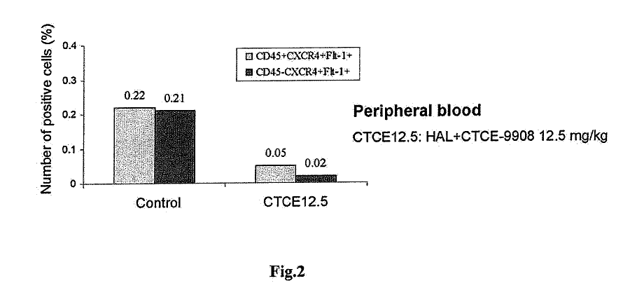

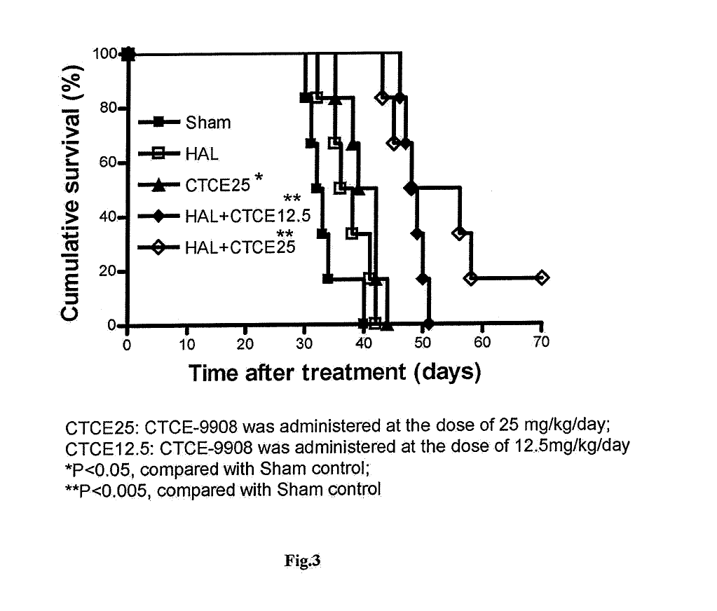

CTCE-9908 treatment was well tolerated by all rats. CTCE-9908 with HAL induced a tumor regression by up to 80% (FIG. 1). A reduction in the circulating hemangiocytes (about 80%-90%) was observed (FIG. 2). This may significantly contribute to the shrinkage of the tumor by inhibiting vasculogenesis and angiogenesis. The survival of rats was correspondingly increased. HAL alone did not change survival compared to sham surgery. CTCE-9908 alone significantly increased survival. Combination of CTCE-9908 with HAL further enhanced survival (FIG. 3).

### Example 2

During the open label, dose-finding trial following the Accelerated Titration Design with 1 subject per site per cohort and following 100% dose increments, not only the safety was demonstrated, but also some efficacy in treating various solid tumors with CXCR4 receptor antagonist compounds. According to the study design, the cohort was to be expanded in the case of moderate toxicity to include more subjects and additional dose increments (40%) until the maximum dose of 5 mg/kg or dose limiting toxicity was achieved. Only one case of moderate toxicity was observed in the 5 mg/kg cohort during the dose escalation and therefore, patients in the standard phase were all treated with 5 mg/kg.

Subjects were dosed daily for 4 consecutive weeks (not including weekends and holidays) and dose limiting toxicity was assessed after subjects have completed their treatment. Subjects who withdrew from the study and did not receive at least 16 doses of study drug were replaced at the same dose level in order to ensure that at least one subject per cohort in the Accelerated Phase had completed treatment.

Toxicity reported during the study was graded according to the National Cancer Institute (NCI) CTCAE (Common Terminology Criteria for Adverse Events) v3.0. A Dose Limiting Toxicity (DLT) was defined as follows:
a) Any grade ≧3 reversible non-haematologic treatment-related toxicity OR
b) Any irreversible grade ≧2 non-haematologic treatment-related toxicity OR
c) Any grade ≧4 haematologic treatment-related toxicity lasting longer than 10 days during the 4 weeks of treatment

For the purpose of this study, Moderate Toxicity has been defined according to NCI CTCAE v3.0. Adverse events were treated when they occurred. No investigational or commercial agents or therapies were administered with the intent to treat the subject's malignancy during the period that the subjects were in the study.

A total of eight Moderate Toxicities were observed during the study while no DLT was reported. Therefore, the Maximum Tolerated Dose (MTD) of CTCE-9908 was not established in subjects with refractory neoplasms during this study. Further dose escalation may determine an MTD. As with many targeted therapies, the MTD may never be reached, and may not be the biologically efficacious dose. However, a number of efficacy trends were observed in this study: overall stable disease in five subjects at the end of their last treatment cycle, including stable disease for up to seven months in one small bowel subject, and reduction in tumor markers in one ovarian cancer subject and one colorectal cancer subject. These were observed at the 1, 2.5 and 5 mg/kg/day doses. These doses may be considered for Phase 2 studies. Future studies may consider the use of CTCE-9908 in combination with other therapies or in other patient populations.

REFERENCES

| Application/Patent Number | Filing Date |
|---|---|
| 11136097/7423011 | May 23, 2005 |
| 09646192 | Mar. 2, 2001 |
| 6,946,445 | Mar. 12, 1999 |
| 09646192 | Mar. 2, 2001 |
| PCT/CA99/00221 | Mar. 12, 1999 |

1: Use of a peptide as a chemokine receptor antagonist, optionally in combination with one or more other chemotherapeutic agent(s) and/or a radiation therapy, in preparation of a medicament for treating cancer or reducing or halting tumor growth in a subject during an embolization therapy,
wherein the peptide specifically binds to human CXCR4.

2: Use of claim 1, wherein the peptide further comprises:
(1) an amino acid sequence represented by SEQ ID NO.1, 2 or 3, or
(2) an amino acid sequence obtained by one or several conservative substitutions in SEQ ID NO.1, 2 or 3.

3: Use of claim 1, wherein the chemotherapeutic agent is at least one selected from the group consisting of an anti-mitotic agent, platinum-based chemotherapeutic agent, receptor tyrosine kinase inhibitor, pyrimidine analogue, topoisomerase inhibitor, and adjuvant.

4: Use of claim 3, wherein the anti-mitotic agent is docetaxel or paclitaxel, or a pharmaceutically acceptable analogue or salt thereof.

5: Use of claim 3, wherein the platinum-based chemotherapeutic agent is cisplatin, carboplatin, iproplatin, or oxaliplatin, or a pharmaceutically acceptable salt thereof.

6: Use of claim 3, wherein the receptor tyrosine kinase inhibitor is sorafenib, sunitinib, or pazopanib, or a pharmaceutically acceptable salt thereof.

7: Use of claim 3, wherein the pyrimidine analogue is gemcitabine, 5-FU, or capecitabine, or a pharmaceutically acceptable salt thereof.

8: Use of claim 3, wherein the topoisomerase inhibitor is irinotecan, topotecan, camptothecin, or lamellarin D, or a pharmaceutically acceptable salt thereof.

9: Use of claim 3, wherein the adjuvant is folinic acid, or a pharmaceutically acceptable salt thereof.

10: Use of claim 3, wherein the chemotherapeutic agent is a combination of 5-FU, folinic acid and oxaliplatin; 5-FU, folinic acid and irinotecan; capecitabine and oxaliplatin; or cisplatin and gemcitabine.

11: Use of claim 1, wherein the radiation is X-rays, gamma rays or charged particles delivered either by a machine outside the body or from a radioactive material placed in the body near the cancer/tumor cells.

12: Use of claim 11, wherein the radioactive material is radioactive iodine.

13: Use of claim 1, wherein the cancer is selected from the group consisting of ovarian cancer, uterus cancer, breast cancer, lung cancer, liver cancer, colorectal cancer, bladder cancer, renal cancer, prostate cancer, pancreatic cancer, stomach cancer, bone cancer, skin cancer, visceral cancers, adrenal and pheochromocytomas, leukemia, and malignant soft tissue sarcoma.

14: Use of claim 1, wherein the peptide and the chemotherapeutic agent are administered concurrently.

15: Use of claim 1, wherein the peptide and the chemotherapeutic agent are administered sequentially.

16: Use of claim 1, wherein the embolization therapy is trans-arterial embolization (TAE), trans-arterial chemoembolization (TACE), or radioembolization (RE).

17. Use of claim 1, wherein the subject is a human.

18: A method of treating cancer or reducing or halting tumor growth in a subject, comprising administering to the subject a peptide, optionally in combination with one or more other chemotherapeutic agents and/or a radiation therapy, during an embolization therapy,
wherein the peptide specifically binds to human CXCR4.

19: The method of claim 18, wherein the peptide further comprises:
(1) an amino acid sequence represented by SEQ ID NO.1, 2 or 3, or
(2) an amino acid sequence obtained by one or several conservative substitutions in SEQ ID NO.1, 2 or 3.
